(19) United States
(12) Patent Application Publication
(10) Pub. No.: US 2008/0213262 A1
(43) Pub. Date: Sep. 4, 2008

(54) Inhibition of Islet Amyloid Polypeptide (Iapp) Aggregation for the Treatment of Type 2 Diabetes

(76) Inventors: Emma Jaikaran, Cambridge (GB); Dawn Bembridge, Cambridge (GB); Jesus Zurdo, Cambridge (GB)

Correspondence Address: KLARQUIST SPARKMAN, LLP, 121 SW SALMON STREET, SUITE 1600, PORTLAND, OR, 97204, US

(21) Appl. No.: 11/883,010

(22) PCT Filed: Jan. 24, 2006
(86) PCT No.: PCT/GB06/00240

§ 371 Date: Jul. 24, 2007

(30) Foreign Application Priority Data
Jan. 24, 2005 (GB) 0501348.7

(51) Int. Cl. A61K 39/00

(52) U.S. Cl. 424/133.1; 424/139.1

(57) ABSTRACT

The present invention relates to the targeting and clearance of soluable unprocessed Islet Amyloid Polypeptide (hIAPP) in order to prevent the nucleation of hIAPP amyloidogenesis and to interfere with pancreatic cell death which is associated with the aggregation of hIAPP. Agents and methods for reducing hIAPP aggregation are provided herein and may be useful in the treatment of type 2 diabetes.

The present invention relates to compositions and methods for treating diseases characterised by secretion of partially or improperly processed protein precursors and by amyloid deposition. In particular the compositions and methods are for the treatment of type 2 diabetes characterised by deposition of islet amyloid polypeptide (IAPP).

Islet amyloid polypeptide (IAPP: also called amylin) is derived from an 89 amino acid precursor protein, pre-proIAPP. After translation, a 22 amino acid signal peptide is cleaved from the N-terminus to give a 67 amino acid proIAPP molecule (Betsholtz et al., 1989, Sanke et al., 1988). A cysteine bridge is formed between amino acids 13 and 18 of the proIAPP molecule. Subsequently, short C- and N-terminal flanking peptides are removed by proteolytic cleavage to give mature (37 amino acid) IAPP. Experiments carried out both in vitro using recombinant or synthetic proIAPP and in mice lacking the gene for either of the processing enzymes (PC1/3, PC2) indicate that cleavage of proIAPP is carried out by the β-cell granule endopeptidases PC1/3 and PC2 (Badman et al., 1996; Higham et al., 2000; Wang et al., 2001; Marzban et al., 2004). Both Pre-proinsulin and proIAPP have been shown to be cleaved by PC1/3 and PC2 and increased levels of partially processed insulin (pro-insulin and des 31,32, proinsulin) and IAPP (including N- and C-Pro fragments) have been noted in human diabetic patients and hIAPP transgenic mice. It has been suggested that a generalised β-cell defect in prohormone processing exists in Type 2 diabetes (Kahn, 1997).

β-cell hyperplasia is found to occur in obese insulin resistant non-type 2 diabetics, and it is believed that this increase in β-cell mass compensates for the increased demand for insulin in non-diabetic insulin resistant individuals In contrast, in type 2 diabetics, decreased β-cell mass is observed. It is thought that in type 2 diabetics cytotoxic aggregation of IAPP is responsible for increased β-cell death via apoptosis (Lorenzo, 1994; MacGibbon, 1997; Tucker, 1998; Janson, 1999; Zhang, 1999; Saafi, 2001; Butler, 2003a; Butler, 2003b).

Using polyclonal antibodies raised against peptides derived from the human sequence of IAPP and the N-flanking peptide of proIAPP, Westermark and colleagues (1989) demonstrated that both mature IAPP and the N-terminus extended form of IAPP (N-terminus unprocessed proIAPP) are found in islet amyloid deposits. N-terminal extended hIAPP (N-Pro-hIAPP) has recently been shown to be increased markedly in cultured human islets after long-term exposure to high glucose, such that it comprises more than half of the total IAPP immunoreactivity (Hou et al., 1999).

Park and Verchere (2001) found that a synthetic peptide corresponding to the first 30 N-terminal amino acids of human proIAPP was able to bind to both heparin and heparan sulphate. Substitution of two basic amino acids lysine (K10) and arginine (R11) with alanine residues abolished this binding ability. The N-terminal 30 amino acid human proIAPP fragment was found not to be fibrillogenic. Park and Verchere speculated that if secretion of unprocessed or partially processed proIAPP is increased in type 2 diabetes, binding to sulphated glycosaminoglycan side chains of heparan sulphate proteoglycans, such as perlecan, might occur. These authors suggest that heparin binding might then induce conformational changes in proIAPP that favour β-sheet formation, enhancing its tendency to aggregate. These aggregates (in the form of amyloid fibrils) would constitute a “nidus” for amyloidogenesis within the pancreatic islet, to which other forms of IAPP could incorporate, giving rise eventually to large amyloid plaques, which according to the authors could constitute an important event in the development of type 2 diabetes. However, no evidence was presented to support this suggestion, and moreover, it has also been suggested that a heparin/heparan sulphate binding interaction might play a normal physiological role, for example in proIAPP trafficking and/or processing (Park and Verchere 2001)

Wang et al., 2001 found that islet amyloid in pancreas of hIAPP transgenic mice is diffuse and uniform affecting all islets before becoming severe. The reduction of islet endocrine cells occurs at the early stages of islet amyloidosis and continues with the progression of diabetes. This underscores the potential importance of arresting islet amyloidosis at its early stages and decreases the relevance of mature amyloid plaques in the progression of the disease.

U.S. Pat. No. 5,643,562 (Kisilevsky) described the use of anionic (primarily sulphated) species to interfere with the interaction between amyloid and glycoproteins in the basement membrane (i.e., Abeta and heparin). However, it was rapidly established (Watson, 1997) that Abeta will only bind to heparin if it is in a fibrillar state, as soluble Abeta does not interact with heparin. Watson and colleagues also demonstrated that mature IAPP must be in a fibrillar state to bind to heparin. Therefore, in U.S. Pat. No. 5,643,562 the interaction observed and targeted is that between fibrillar forms of amyloid and glycosaminoglycans and glycoproteins in the basement membrane.

No inhibitors of hIAPP cytotoxicity or amyloid formation are currently available to treat type 2 diabetes. Several compounds under development are concerned with inhibiting fibril formation, the end stage of amyloidosis, rather than inhibition of the early nucleating steps with a view to prevent the formation of cytotoxic early aggregates. Various attempts to inhibit the interaction of amyloidogenic proteins with extracellular matrix components are currently being pursued (WO03017994, WO2005000288, WO0242429, US2001055630 and related patents). However, although these compounds target mature hIAPP aggregation, or the stability of amyloid fibrils formed by mature hIAPP, their interaction with the basement membrane, and some report to be able to dissociate amyloid fibrils, they do not address the very early steps in the amyloidogenic process that initiate in the nucleation event that trigger the formation of early cytotoxic aggregates, which seem to be responsible for the beta-cell death reported in type II diabetes (Janson, 1996; Janson, 1999; Kayed, 2003).

There is a need for selective therapies that target early events in type II diabetes to prevent initiation as well as to reverse progression of amyloidosis. The present inventors have addressed this need by specifically targeting soluble unprocessed proIAPP or soluble partially processed forms of IAPP that include the N-terminal fragment of proIAPP, to remove it from circulation before it binds to the basement membrane to prevent nucleation and amyloid fibril formation by mature IAPP.

Unprocessed soluble proIAPP, or partially processed soluble N-terminal intact proIAPP is shown herein to be responsible for the initiation of islet amyloid formation by facilitating a nucleation event. By targeting this early form of proIAPP this nucleation event (stochastic process) can be substantially impaired through this strategy, which in turn will reduce the accumulation of early aggregates of IAPP. Such early aggregates constitute the cytotoxic species responsible for the pancreatic β-cell death associated to type II diabetes (Janson, 1996; Janson, 1999; Kayed, 2003). Therefore, interference with the early events driving the hIAPP aggregation process will deter β-cell death, halting the progression from insulin resistance to diabetes.

An aspect of the present invention provides agents, including specific ligands or antibodies or other specific binding members, targeted against unprocessed or improperly processed soluble human proIAPP containing the N-terminal segment of proIAPP (N-Pro fragment). Such agents may increase the clearance of such abnormally processed soluble species This decreases the probability of an amyloid nucleating event, which in turn prevents the formation of cytotoxic aggregates of IAPP in pancreas reducing β-cell apoptosis and delaying the onset of diabetic symptoms in patients.

In some embodiments, agents which bind specifically to unprocessed or improperly processed soluble human proIAPP may prevent or reduce the binding of abnormally processed soluble human proIAPP to glycoproteins in the basal membrane. In other embodiments, agents which bind specifically to unprocessed or improperly processed soluble human proIAPP may have no effect or substantially no effect on the binding of abnormally processed soluble human proIAPP to glycoproteins in the basal membrane.

By clearing or removing unprocessed or improperly processed forms of IAPP, amyloid deposition and cytotoxicity and β-cell destruction associated with amyloid deposition may be reduced substantially or even be avoided completely. This would remove the necessity for agents targeted to decrease amyloid fibril load or interacting with IAPP, which could have a pernicious effect in controlling the glucose levels in blood.

By targeting the earliest steps in the aggregation pathway, involving unprocessed or partially processed proIAPP, early aggregates are prevented from forming and thus cytotoxic activity stopped, allowing β-cell mass to increase and cope with increased insulin requirement. By increasing the clearance of proIAPP or mis-processed proIAPP (including N-Pro-IAPP), and, in some embodiments, preventing interaction between proIAPP or mis-processed proIAPP (including N-Pro-IAPP) and extracellular glycosamineglycans, the formation of a nucleus for amyloidogenesis is prevented. As amyloid formation is a nucleation-dependent phenomenon, this strategy interferes with the necessary nucleation step for the formation of both diffuse and fibrillar deposits of islet amyloid. In addition, this approach disrupts the formation of small oligomeric species of hIAPP which have been shown to be the cytotoxic species responsible for islet β-cell death (Janson, 1996; Janson, 1999; Kayed, 2003). By preventing initiation of amyloid deposition, not only may islet amyloid deposition be reduced or prevented, but cytotoxicity due to early soluble aggregates will also be reduced or abolished.

Another aspect of the invention provides an agent which is capable of binding specifically to a soluble precursor of an amyloidogenic protein or peptide.

The amyloidogenic protein is preferably human IAPP and the soluble precursor is preferably an unprocessed or improperly processed soluble human proIAPP containing the N-terminal segment of proIAPP (N-Pro fragment: SEQ ID NO:5), for example N-proIAPP (SEQ ID NO:2) and/or proIAPP (SEQ ID NO:3).

By ‘binding specifically’ is meant that the agent binds to the precursor and not other proteins present in a sample or organism, such as the mature amyloidogenic protein or peptide. Specific binding to the precursor molecules resulting in their clearance reduces, inhibits or prevents nucleation of amyloid.

The agent may be a specific binding member such as a peptide, peptide analogue, antibody or antibody fragment. In some embodiments, the agent may also prevent or interfere with the binding to heparin and may be used to inhibit binding of the soluble protein or peptide to the basement membrane proteoglycan.

Also provided are agents, including specific binding members, which are capable of specifically binding to, and, optionally, of specifically inhibiting the interaction with a basement membrane heparan sulphate proteoglycan of, one or more of soluble proIAPP (SEQ ID NO: 2), N-Pro-hIAPP (SEQ ID NO: 3), or peptide fragments thereof containing the N-Pro sequence (SEQ ID NO: 5), such as N-proKC (SEQ ID NO: 1). In some embodiments, the agent may be capable of binding specifically to an epitope on the heparan sulphate binding site of a polypeptide comprising the N-Pro sequence (SEQ ID NO: 5) or N-ProKC sequence (SEQ ID NO: 1). An agent which binds specifically to one or more of soluble proIAPP (SEQ ID NO: 2), N-Pro-hIAPP (SEQ ID NO: 3), or peptide fragments thereof containing the N-Pro sequence (SEQ ID NO: 5), does not bind to mature hIAPP (SEQ ID NO: 4).

In some embodiments, an agent may bind specifically to one or more of soluble N-Pro sequence (SEQ ID NO: 5), N-proKC (SEQ ID NO: 1), proIAPP (SEQ ID NO: 2), N-Pro-hIAPP (SEQ ID NO: 3) and N-Pro30 (SEQ ID NO: 6) but not to mature hIAPP (SEQ ID NO: 4).

The term “antibody” as used herein includes, but is not limited to: polyclonal, monoclonal, bispecific, humanised or chimeric antibodies, single chain antibodies, Fab fragments and F (ab′)₂ fragments, fragments produced by a Fab expression library, anti-idiotypic (anti-Id) antibodies, and, proIAPP (SEQ ID NO: 2) or N-Pro-hIAPP (SEQ ID NO: 3) binding fragments of any of the above. The term “antibody” as used herein also refers to immunoglobulin molecules and immunologically-active portions of immunoglobulin molecules, i.e. molecules that contain a binding site that specifically binds proIAPP (SEQ ID NO: 2) and/or N-Pro-hIAPP (SEQ ID NO: 3) and polypeptides containing the N-Pro sequence (SEQ ID NO: 5), including, for example, N-Pro30 (SEQ ID NO:6). The immunoglobulin molecules of the invention can be of any class (e.g., IgG, IgE, IgM, IgD and IgA) or subclass of immunoglobulin molecule.

The antibody may be selected from the group consisting of: a human antibody, a rodent antibody, a murine antibody, a camelid antibody, a recombinant human antibody, a humanised murine antibody, a chimerised murine antibody, a transgenic murine antibody and a chimerised or humanised camelid antibody.

Other suitable specific binding members include affibodies or affinity peptides or proteins, which may be isolated or fused to a scaffold proteinic component.

Other aspects of the invention relate to the identification of antibodies and antibody antigen-binding domains which specifically bind to one or more of, soluble proIAPP (SEQ ID NO: 2) N-Pro-hIAPP (SEQ ID NO: 3), and peptide fragments containing the N-Pro sequence (SEQ ID NO: 5), such as N-ProKC, (SEQ ID NO: 1) and may therefore be useful in inhibiting hIAPP aggregation, for example in the treatment of type 2 diabetes.

A method of producing an antibody may comprise:
administering an immunogen comprising proIAPP (SEQ ID NO: 2), N-Pro-hIAPP (SEQ ID NO: 3), or a peptide fragment thereof comprising the N-Pro sequence (SEQ ID NO: 5), such as N-ProKC (SEQ ID NO: 1), N-Pro (SEQ ID NO: 5) or N-pro30 (SEQ ID NO:6) as described herein to an animal, and;
isolating from said animal an antibody which binds to said immunogen.

The antibody may specifically bind to soluble proIAPP (SEQ ID NO: 2) and/or N-Pro-hIAPP (SEQ ID NO: 3), and may optionally inhibit the binding of proIAPP to heparin.

The binding of an antibody isolated from an animal to hIAPP may be determined to identify antibodies which bind to proIAPP precursors but not mature IAPP.

A molecule which binds specifically shows no significant binding to molecules other than its specific binding partner(s). Where, for example, an antigen-binding site is specific for a particular epitope, the specific binding member carrying the antigen-binding site will be able to bind to the various molecules carrying the particular epitope. For example, an antibody antigen-binding domain specific for an IAPP precursors such as N-proIAPP may show binding to other molecules carrying the same epitope, which may include proIAPP, or peptide fragments thereof comprising the N-pro sequence. An antibody antigen-binding domain specific for IAPP precursors such as proIAPP and N-proIAPP as described herein may show no binding or substantially no binding to the mature hIAPP sequence. Preferably. a suitable antibody antigen-binding domain does not cross-react with rodent IAPP precursors, for example murine proIAPP)

The immunogen may comprise a protein carrier, such as Keyhole Limpet Haemocyanin. Other suitable carriers are well known in the art.

Antibodies may be obtained from immunised animals using any of a variety of techniques known in the art, and screened, preferably using binding of antibody to antigen of interest. For instance, Western blotting techniques or immunoprecipitation may be used (Armitage et al. (1992) Nature 357 80-82).

More preferably, an antibody molecule may be a monoclonal antibody. Methods of producing monoclonal antibodies are well known in the art (see, for example, Harlow et al Antibodies: A Laboratory Manual, Cold Spring Harbor Laboratory (Cold Spring Harbor, N.Y., 1988) pp. 353-355) and are described in more detail below. For example, antibody-producing cells may be isolated from an immunised mammal and fused with immortalised cells to produce a population of antibody-producing hybridoma cells, which can then be screened to identify a hybridoma cell that produces an antibody which displays optimal binding characteristics.

In some embodiments, a hybridoma may be produced by a method comprising;
immunizing a non-human mammal with an immunogen comprising proIAPP (SEQ ID NO: 2), N-Pro-hIAPP (SEQ ID NO: 3), or a peptide fragment thereof comprising the N-Pro sequence (SEQ ID NO: 5), such as N-ProKC (SEQ ID NO: 1), N-Pro (SEQ ID NO: 5) or N-pro30 (SEQ ID NO:6),
producing one or more fusions of antibody producing cells from said mammal and immortalised cells to provide a population of hybridoma cells, and;
screening said population to identify a hybridoma cell which produces an antibody which binds the immunogen.

The population of hybridoma cells is preferably screened by testing the binding of antibodies produced by cells of the population to an IAPP precursor such as soluble proIAPP (SEQ ID NO: 2) or N-Pro-hIAPP (SEQ ID NO: 3) or a fragment thereof such as N-ProKC (SEQ ID NO: 1), N-Pro sequence (SEQ ID NO: 5) or N-pro30 (SEQ ID NO:6). Conventional techniques such as western blotting or immunoprecipitation may be used. The population of hybridoma cells may be further screened by testing the binding of antibodies produced by cells of the population to mature hIAPP (SEQ ID NO:4). Preferably, antibodies produced by cells of the population show little or no binding to mature hIAPP (SEQ ID NO:4).

Hybridoma cells identified as producing antibodies which bind to an IAPP precursor such as soluble proIAPP (SEQ ID NO: 2) or N-Pro-hIAPP (SEQ ID NO: 3) or a fragment thereof such as N-ProKC (SEQ ID NO: 1), N-Pro sequence (SEQ ID NO: 5) or N-pro30 (SEQ ID NO:6) but do not bind to mature hIAPP (SEQ ID NO:4 may be isolated and/or purified from the population.

Following isolation, the hybridoma may be expanded, maintained and/or cultured in a culture medium using methods which are well-known in the art. Antibodies produced by the hybridoma may be isolated from said culture medium.

A method of producing an antibody may comprise;
culturing a hybridoma cell produced as described above in a culture medium; and,
isolating from the medium an antibody as described above, for example, an antibody which binds to an IAPP precursor such as soluble proIAPP (SEQ ID NO: 2) or N-Pro-hIAPP (SEQ ID NO: 3) or a fragment thereof such as N-ProKC (SEQ ID NO: 1), N-Pro sequence (SEQ ID NO: 5) or N-pro30 (SEQ ID NO:6) but does not bind to mature hIAPP (SEQ ID NO:4).

Alternatively, a monoclonal antibody specific for a peptide, polypeptide or peptidyl trimer as described herein may be obtained from a recombinantly produced library of expressed immunoglobulin variable domains or other molecules comprising antibody antigen-binding domains, e.g. using lambda bacteriophage or filamentous bacteriophage which display functional immunoglobulin binding domains on their surfaces; for instance see WO92/01047. The library may be immunologically naive, that is constructed from sequences obtained from an organism which has not been immunised with a peptide comprising the epitope, or may be one constructed using sequences obtained from an organism which has been exposed to the antigen of interest.

A method of producing an antibody may comprise:
contacting a peptide comprising or consisting of proIAPP (SEQ ID NO: 2), N-Pro-hIAPP (SEQ ID NO: 3), or a peptide fragment thereof comprising or consisting of the N-Pro sequence (SEQ ID NO: 5), such as N-ProKC (SEQ ID NO: 1), N-Pro (SEQ ID NO: 5) or N-pro30 (SEQ ID NO:6) with a diverse population of antibody antigen-binding domains, and;
determining the binding of members of said population to said peptide.

The antibody antigen-binding domains may be comprised in antibodies or scFv, Fab, Fv, dAb, Fd or diabody molecules.

An antibody antigen-binding domain may be identified in said population which binds to the peptide.

Antibody antigen-binding domains may be displayed on the surface of virus particles i.e. the diverse population may be a phage display library.

The virus particle which displays the identified antibody antigen-binding domain may be isolated and/or purified and the nucleic acid encoding the antibody antigen-binding domain obtained from said particle.

The nucleic acid encoding the antibody antigen-binding domain may be sequenced and/or expressed to produce the encoded antibody antigen-binding domain that binds to the peptide.

An antibody antigen-binding domain produced as described above may be further tested using routine methodology to determine its specificity.

In some embodiments, the binding properties of the antibody antigen-binding domain may be further optimised using standard antibody engineering techniques, including affinity maturation, for example by chain shuffling, and site-specific, random or combinatorial mutagenesis.

An antibody antigen-binding domain which is comprised in an antibody molecule, for example an antibody, scFv, Fab, Fv, dAb, Fd or diabody molecule, may be reformatted, for example into an IgG antibody, using standard techniques for subsequent use.

The antibody molecule or specific binding member may be tested for anti-hIAPP aggregation activity. For example, the ability of the antibody molecule or specific binding member to reduce to inhibit IAPP aggregation may be determined.

An antibody molecule or specific binding member which has anti-hIAPP aggregation activity may be formulated into a pharmaceutical composition, for example by admixing with a pharmaceutical carrier, as described herein.

Agents or antibodies as described herein are capable of binding specifically to N-Pro-hIAPP (SEQ ID NO: 3) and polypeptides comprising N-pro-hIAPP, such as proIAPP (SEQ ID NO: 2). As discussed above, such agents or antibodies show little or no binding to mature hIAPP (SEQ ID NO:4).

Preferably the agent or antibody is capable of binding specifically to a soluble protein or peptide consisting of, or comprising, an amino acid sequence selected from:
a) the amino acid sequence of SEQ ID NO: 1;
b) residues 1-11 of the sequence of SEQ ID NO: 1 (SEQ ID NO: 5);
c) fragments of 5-8 amino acids of the N-terminal sequence of SEQ ID NO: 1;
d) fragments of 5-8 amino acids of the sequence of SEQ ID NO: 1 including His6, Lys10 Arg11 and Lys12, and
(e) fragments of 5-8 amino acids of the sequence of SEQ ID NO: 3 which include 1, 2, 3, 4, 5 or more residues of SEQ ID NO:5.

The binding peptide could be part of a fusion protein, with a carrier or other conjugate peptide moiety, or could be conjugated to another molecule, such as a sugar moiety, a lipid, a nucleotide, a nucleic acid, a hormone, a modifying group including an aliphatic or aromatic group, an unnatural amino acid, a surfactant, a polymer, an artificial matrix, or a fluorescent marker.

An agent according to the invention could be a synthetic chemical molecule, or naturally or recombinantly produced biological molecule, identifiable by screening against soluble N-Pro (SEQ ID NO: 5) or a suitable fragment thereof, or against a peptide including soluble N-Pro (SEQ ID NO: 5), such as N-proKC (SEQ ID NO:1), proIAPP (SEQ ID NO: 2), N-Pro-hIAPP (SEQ ID NO: 3) or N-Pro30 (SEQ ID NO:6), or suitable fragment thereof.

The invention also provides agents capable of stimulating production of an immune response in an individual to whom the agent has been administered, said immune response being capable of inhibiting specifically the interaction between a soluble precursor of an amyloidogenic protein or peptide and a basement membrane proteoglycan.

The N-terminal sequence may be used to target anti-amyloidogenic agents to the extracellular space/amyloid deposits; hence also provided are chimeric peptides comprising a) the amino acid sequence of SEQ ID NO: 1; b) residues 1-11 of the sequence of SEQ ID NO: 1 (SEQ ID NO: 5); c) fragments of 5-8 amino acids of the N-terminal sequence of SEQ ID NO: 1; or d) fragments of 5-8 amino acids of the sequence of SEQ ID NO: 1 including His6, Lys10 Arg11 and Lys12. Such a peptide could be used as a drug delivery system for anti-amyloidogenic drugs, since in several systemic amyloidoses the deposition of amyloid is linked to glycosamineglycans of the basement membrane in various organs (i.e. AA or AL amyloidoses); other non-systemic amyloidoses could perhaps be targeted by this (i.e. Alzheimer's disease).

Chimeric peptides may be generated wholly or partly by chemical synthesis. The peptides can be readily prepared, for example, according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984); in J. H. Jones, The Chemical Synthesis of Peptides. Oxford University Press, Oxford 1991; in Applied Biosystems 430A Users Manual, ABI Inc., Foster City, Calif., in G. A. Grant, (Ed.) Synthetic Peptides, A User's Guide. W. H. Freeman & Co., New York 1992, E. Atherton and R. C. Sheppard, Solid Phase Peptide Synthesis, A Practical Approach. IRL Press 1989 and in G. B. Fields, (Ed.) Solid-Phase Peptide Synthesis (Methods in Enzymology Vol. 289). Academic Press, New York and London 1997), or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

Another convenient way of producing a peptide as described herein (peptide or polypeptide) is to express nucleic acid encoding the peptide using routine recombinant techniques.

The peptide could be provided as a fusion protein/conjugated to another moiety which could be a peptide or other molecule, either acting as a carrier and/or as an adjuvant to stimulate immune response and/or to protect the peptide from degradation. A fusion protein may be seroalbumin, B-cell ligand, etc. any other form of targeting; conjugation may include BSA, HRP, etc; adjuvants such as Freunds, Ribi Adjuvant, Titermax, Alum, may be used; and stabilisers such as glycation, acylation, use of polyethylene glycol and derivatives, etc. any other form of encapsulation of delivery.

Structural and peptide mimics of particular epitopes are also provided, as are nucleic acids encoding peptides and agents of the present invention.

Agents, antibodies and/or other specific binding members of the invention may be used in the manufacture of medicaments for treatment of amyloidogenic disorders, or as pharmaceutical compositions. In particular, agents and/or antibodies may be used in the treatment of type 2 diabetes. A method of treatment may, for example, comprise administration of such a pharmaceutical composition to a patient, e.g. for a therapeutic purpose, which may include preventative treatment, and a method of making a pharmaceutical composition comprising admixing such a agent with a pharmaceutically acceptable excipient, vehicle or carrier, and optionally other ingredients.

A pharmaceutically useful compound according to the present invention that is to be given to an individual, is preferably administered in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

A composition may be administered alone or in combination with other treatments, either simultaneously or sequentially dependent upon the condition to be treated.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Liposomes, particularly cationic liposomes, may be used in carrier formulations. In other embodiments, an agent as described herein may be coupled to inert polymer support.

Examples of techniques and protocols mentioned above can be found in Remington's Pharmaceutical Sciences, 16th edition, Osol, A. (ed), 1980.

An agent or pharmaceutical composition may be administered alone or in combination with other treatments, either simultaneously or sequentially dependent upon the condition to be treated.

Other aspects of the invention relate to methods of identifying an agent capable of binding to soluble molecules corresponding to unprocessed or partially processed forms of proIAPP (SEQ ID NO: 2) that include the sequence N-Pro (SEQ ID NO: 5) or N-proKC (SEQ ID NO:1) or fragments thereof.

A method of screening for an agent which specifically binds to unprocessed or partially processed forms of proIAPP (SEQ ID NO: 2) that include the sequence N-Pro (SEQ ID NO: 5) and which may for example be useful in the treatment of type II diabetes, may comprise:

contacting a test compound with a proIAPP polypeptide with a test compound, and
determining the binding of the test compound to the proIAPP polypeptide.

The test compound may be contacted with a IAPP polypeptide of SEQ ID NO: 4 and the binding of the test compound to the IAPP polypeptide determined.

A test compound which binds to the proIAPP polypeptide but not the IAPP polypeptide may be useful in the treatment of type II diabetes.

Another aspect of the invention provides method of screening for an agent which inhibits the binding of unprocessed or partially processed forms of proIAPP (SEQ ID NO: 2) that include the sequence N-Pro (SEQ ID NO: 5) or N-proKC (SEQ ID NO:1) or fragments thereof, to heparan sulphate moieties of extracellular glycosamineglycans, and which may for example be useful in the treatment of type II diabetes, the method comprising:
contacting a glycosamineglycan comprising one or more heparan sulphate moieties with a proIAPP polypeptide in the presence of a test compound and,
determining the binding of the glycosamineglycan and the proIAPP polypeptide.

A decrease in binding in the presence of the test compound is indicative that the test compound is a putative agent for use in the treatment of type II diabetes.

A proIAPP polypeptide is preferably a polypeptide which has the sequence of SEQ ID NO: 2 or SEQ ID NO: 3 or a peptide fragment thereof which comprises the N-Pro sequence (SEQ ID NO: 5), such as N-ProKC (SEQ ID NO: 1), N-Pro (SEQ ID NO: 5) or N-pro30 (SEQ ID NO:6. In other embodiments a proIAPP polypeptide may be a variant of one of these sequences.

A variant of proIAPP polypeptide is immunologically cross reactive with wild type proIAPP polypeptide and may comprise an amino acid sequence which shares greater than about 80% sequence identity with proIAPP (SEQ ID NO: 2), N-Pro-hIAPP (SEQ ID NO: 3) or N-Pro30 (SEQ ID NO:6), greater than about 90% or greater than about 95%.

Sequence identity is commonly defined with reference to the algorithm GAP (Genetics Computer Group, Madison, Wis.). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Nucl. Acids Res. (1997) 25 3389-3402) may be used. Sequence identity and similarity may also be determined using Genomequest™ software (Gene-IT, Worcester Mass. USA). Sequence comparisons are preferably made over the full-length of the relevant sequence described herein.

The amount of test substance or compound which may be added to a method described herein will normally be determined by trial and error depending upon the type of compound used. Typically, from about 0.01 to 100 nM concentrations of putative inhibitor compound may be used, for example from 0.1 to 10 nM.

A test compound suitable for use in the present methods may be a small chemical entity, peptide, antibody molecule or other molecule whose effect on proIAPP/heparin sulphate binding is to be determined. Natural or synthetic chemical compounds may be used, or extracts of plants which contain several characterised or uncharacterised components.

Suitable test compounds may be selected from compound collections and designed compounds. Combinatorial library technology (Schultz, JS (1996) Biotechnol. Prog. 12:729-743) provides an efficient way of testing a potentially vast number of different substances for ability to proIAPP/heparan sulphate interaction.

Other suitable test compounds include molecules comprising antibody antigen binding domains. For example, libraries of antibody antigen binding domains displayed on virus particles may be screened to identify an antibody antigen binding domain which decreases or inhibits the proIAPP/heparin sulphate interaction.

Methods of identifying a compound or an agent capable of binding to soluble molecules corresponding to unprocessed or partially processed forms of proIAPP (SEQ ID NO: 2) that include the sequence N-Pro (SEQ ID NO: 5) or N-proKC (SEQ ID NO:1) or fragments thereof may use any or all of the following techniques: Immunoassays (including blotting, immunoprecipitation, ELISA-related, radioimmunoassay, immunohistochemistry, etc.), affinity chromatography, binding assays based on surface plasmon resonance, cross-linking, spectroscopic methods (fluorescence spectroscopy, UV-Visible and infrared spectroscopy, circular dichroism, nuclear magnetic resonance, X-ray diffraction, etc.), biochemical assays, cell-based assays, histological assays, animal studies, etc.

The effect of a compound identified by a method described above may be assessed in a secondary screen. For example, the effect of the compound on hIAPP aggregation or β-cell cytotoxicity may be determined. Secondary screens may be performed in in vitro test systems or in vivo in animal models.

Such methods are suitable for identifying agents that are capable of preventing or treating type 2 diabetes. A method as described herein may comprise identifying a test compound as an agent which specifically binds to an IAPP precursor such as proIAPP (SEQ ID NO:2) or N-proIAPP (SEQ ID NO:3) and therefore increases the clearance of IAPP precursors and reduces hIAPP aggregation activity and may be useful in the treatment of type 2 diabetes.

The identified compound may be isolated and/or purified. In some embodiments, the compound may be prepared, synthesised and/or manufactured using conventional synthetic techniques.

Optionally, compounds identified as agents which specifically bind to unprocessed or partially processed forms of proIAPP (SEQ ID NO: 2) and/or inhibit the binding of unprocessed or partially processed forms of proIAPP (SEQ ID NO: 2) to heparan sulphate moieties of extracellular glycosamineglycans using an method described herein may be modified or subjected to rational drug design techniques to optimise activity or provide other beneficial characteristics such as increased half-life or reduced side effects upon administration to an individual.

Compound produced by the screening methods and/or drug design methods described above may be formulated into a composition, such as a medicament, pharmaceutical composition or drug, with a pharmaceutically acceptable excipient.

Controls are employed as appropriate within the routine knowledge and expectation of those skilled in the art.

Various further aspects and embodiments of the present invention will be apparent to those skilled in the art in view of the present disclosure, including the following experimentation to illustrate embodiments of the invention and the accompanying figures.

All documents mentioned in this specification are incorporated herein by reference in their entirety.

The term “comprises” as used herein encompasses both “includes”, i.e. permitting the presence of one or more additional components and “consists of” i.e. not permitting the presence of one or more additional components.

All peptide structures and sequences are indicated using the standard amino acid single letter code.

Certain aspects and embodiments of the invention will now be illustrated by way of example and with reference to the figures described below.

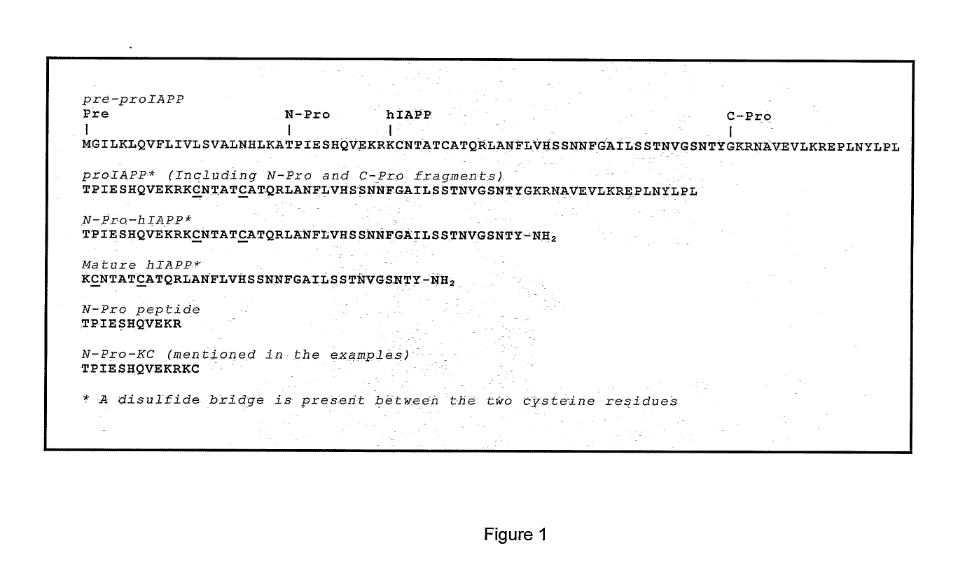

FIG. 1 shows the amino acid sequences of hIAPP and precursors.

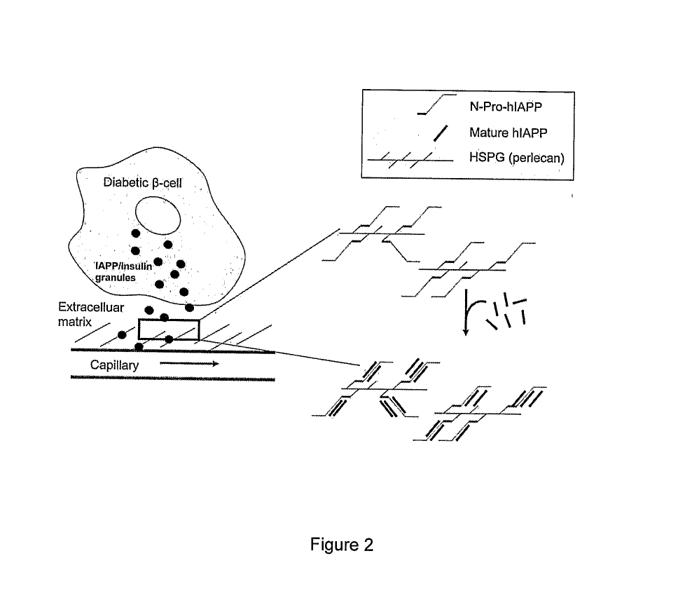

FIG. 2 shows scheme of mature hIAPP amyloid nucleation facilitated by the interaction of misprocessed (N-terminal extended) forms of hIAPP bound to glycosamineglycans (N-Pro-hIAPP in the drawing). N-Pro-hIAPP binds to heparin, rendering an extracellular matrix suitable for amyloid nucleation. Mature hIAPP binds to immobilised N-Pro-hIAPP, nucleating amyloid deposition. The interaction of N-pro region of proIAPP with Heparin/Heparan sulphate groups of perlecan thus results in nucleation of hIAPP amyloid deposits.

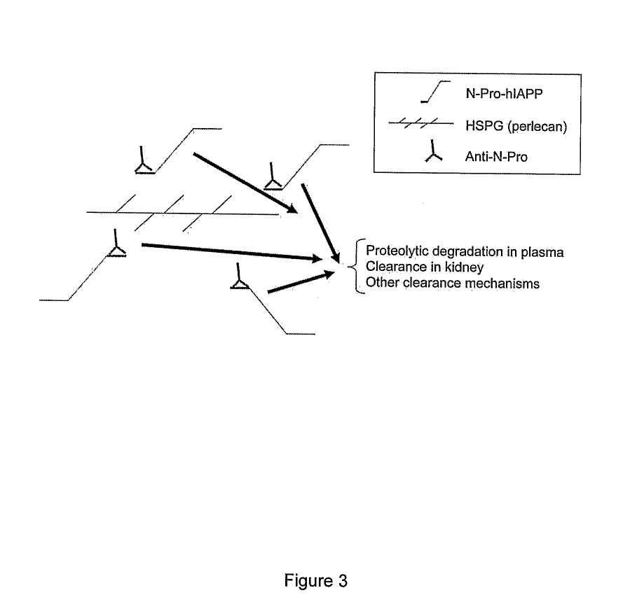

FIG. 3 shows an example of intervention using an anti-N-Pro antibody. The anti-N-Pro antibody clears the N-pro-hIAPP from the bloodstream preventing the nucleation of hIAPP amyloid deposits, The anti-N-Pro antibody may also block the interaction between N-pro-hIAPP and proteoglycans of the basement membrane. N-pro-hIAPP bound to the anti-N-Pro Ab may be eliminated through normal clearance mechanisms, including proteolytic degradation in the plasma and clearance in the kidney. Amyloid nucleation is prevented and there is a reduction in hIAPP aggregate mediated β-cell toxicity. Interference with deposition in the extracellular space via proteoglycan interaction may also facilitate clearance of unprocessed N-pro-hIAPP.

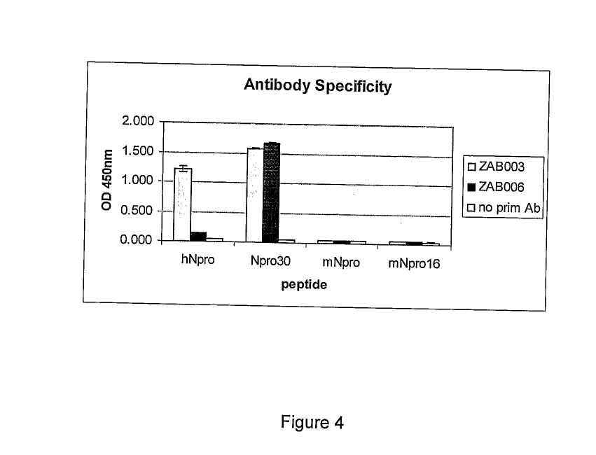

FIG. 4 shows the results of ELISA experiments showing the binding of the ZAB003 and ZAB006 monoclonal antibodies to human and mouse N-pro and N-pro30 peptides.

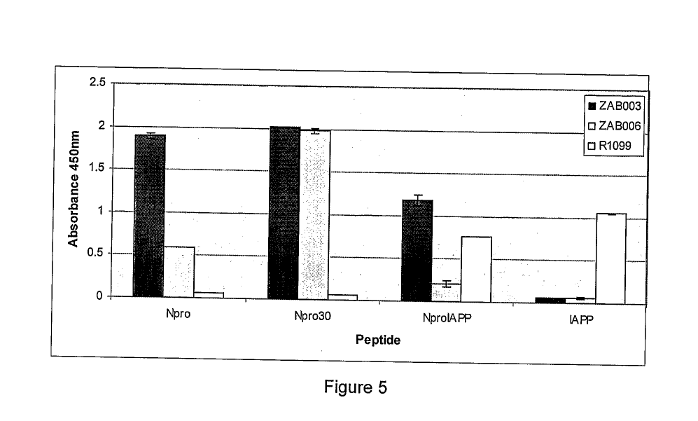

FIG. 5 shows the results of ELISA experiments showing the binding of the ZAB003 and ZAB006 monoclonal antibodies to human N-proIAPP and IAPP peptides.

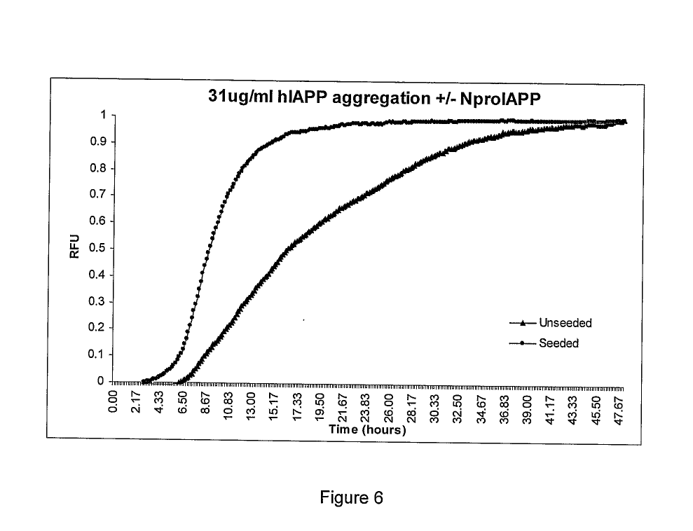

FIG. 6 shows the aggregation profile of 31 ug/ml hIAPP aggregation with and without NproIAPP.

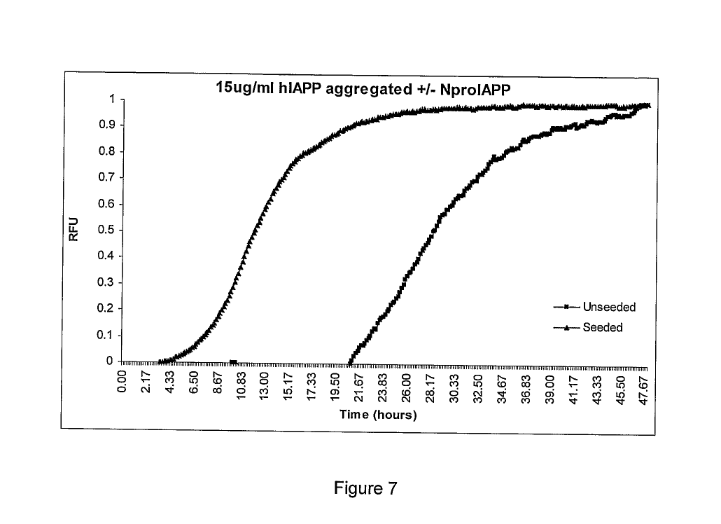

FIG. 7 shows the aggregation profile of 15 ug/ml hIAPP aggregation with and without NproIAPP.

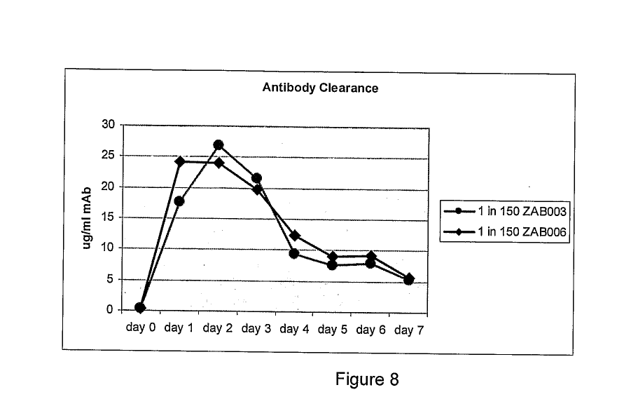

FIG. 8 shows the clearance of ZAB003 and ZAB006 antibodies from hIAPP^{+/+} transgenic C57BI/6J black mice

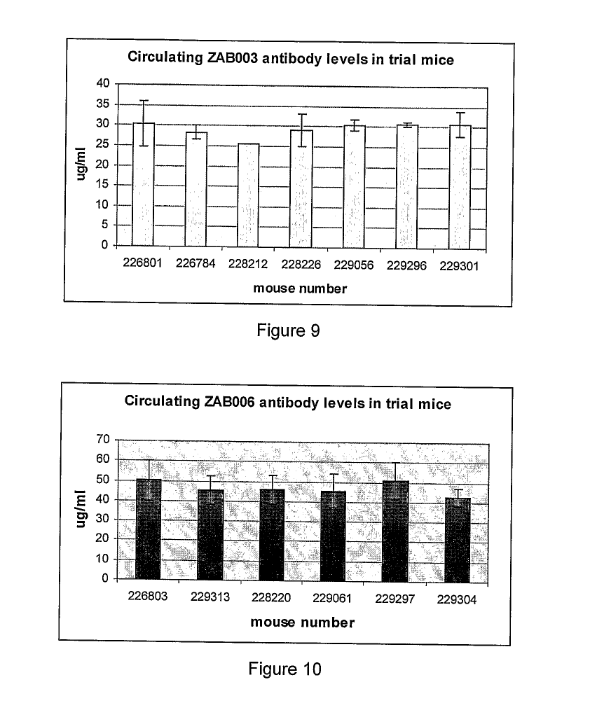

FIG. 9 shows the level of circulating ZAB003 in trial mice after eight weeks of treatment

FIG. 10 shows the level of circulating ZAB006 in trial mice after eight weeks of treatment

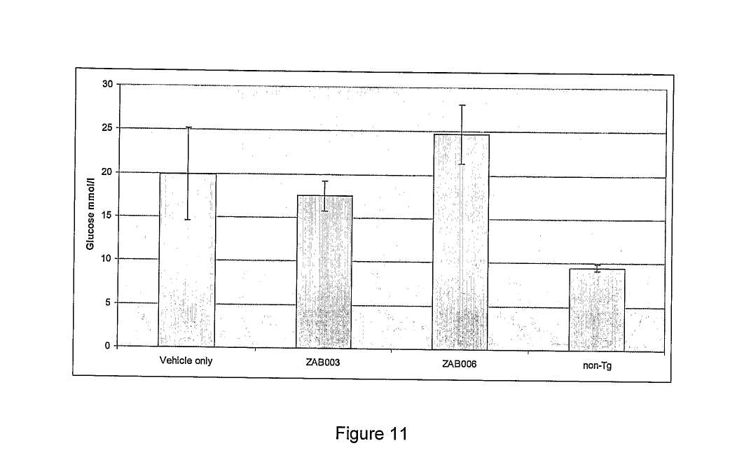

FIG. 11 shows fasting glucose levels in mice after eight weeks of treatment with either ZAB003 or ZAB006

### EXAMPLES

### Example 1

### Synthesis of a Fragment of proIAPP, N-Pro-KC, and Production of Monoclonal Antibodies Against it

The N-Pro-KC peptide (TPIESHQVEKRKC, 13 amino acids, SEQ ID NO: 1) was synthesised using standard Fmoc chemistry. The peptide was conjugated to BSA (Bovine Serum Albumin) via the C-terminus cysteine residue, and the peptide conjugate used to immunize mice to raise antisera. Three to five mice were injected with the N-Pro-KC conjugate antigen. Following immunisation, tail bleeds were taken from the mice at regular intervals of 1-2 weeks and tested for recognition against the N-Pro-KC peptide by ELISA. Boost injections of antigen were then administered, depending on the antibody titre observed by ELISA. B cells extracted from spleen of mice showing a positive response against the antigen were fused to myeloma cells to generate a collection of hybridomas and the clones are expanded for further screening. A total of five hundred clones were screened by ELISA including a negative control of mature hIAPP which should not be recognised by the selected clones. Thirty-three positive clones were re-evaluated using additional ELISAs and five supernatants submitted to further testing as described.

### Example 2

### Specificity of Monoclonal Antibodies Against the N-Pro (SEQ ID NO: 5) Sequence

Interaction of monoclonal antibodies and N-Pro30 (N-Pro plus first 19 residues of mature hIAPP; SEQ ID NO: 6) can be measured using a standard direct ELISA protocol. Briefly, the monoclonal antibody, N-Pro peptide or N-Pro30 peptide is coated onto an ELISA plate, followed by exposure to either N-Pro30 or monoclonal respectively. Binding is detected by the use of an appropriate HRP conjugate and monitored by a standard TMB absorbance protocol.

An ELISA was performed to determine the specificity of two monoclonal antibodies ZAB003 and ZAB006, using the following peptides:

(SEQ ID NO:5)

| | |
|---|---|
| human Npro | TPIESHQVEKR |

(SEQ ID NO:6)

| | |
|---|---|
| Npro30 | TPIESHQVEKRKCNTATCATQRLANFLVHS |

(SEQ ID NO:7)

| | |
|---|---|
| Mouse Npro | TPVRSGSNPQMDKR |

(SEQ ID NO:8)

| | |
|---|---|
| Mouse Npro16 | TPVRSGSNPQMDKRKC |

200 ug lyophilised peptide was resuspended in 200 ul 5 mM TCEP (Sigma: made to 5 mM in water and filtered through 0.22 μm filter and stored −20° C.). Peptide was diluted 1:1000 with carbonate coating buffer (50 mM Sodium Bicarbonate coating buffer pH9.6), and 100 ul/well added to Immulon 96 well maxisorp plate (Nunc). The plate was then incubated overnight at 4° C. and washed three times with Wash Buffer (PBS/0.05% Tween). Monoclonal antibody was added at 0.25 ug/well in 100 ul diluent (3% BSA in PBS/0.01% Tween) and the plate incubated for 1.5 hours at room temp, followed by three washes with Wash Buffer. Goat anti mouse HRP secondary antibody was added at 1:5000 in diluent, followed by incubation at room temp for 1 hour. The plate was then washed four times with Wash Buffer and the ELISA developed by adding 100 ul/well TMB substrate (Sigma) for 5 minutes. The reaction was stopped with the addition of 25 ul of 2M Sulphuric acid and absorbance read at 450 nm.

Results are shown in FIG. 4. ZAB003 was shown to bind both human Npro and Npro30 peptides, but not to bind to the mouse peptides. ZAB006 was shown to bind only to Npro30, and not to human Npro or the mouse peptides. This indicates that the epitope for ZAB006 is not, at least in its entirety, within the first eleven amino acids of Npro.

To further characterise the specificity of the monoclonal antibodies ZAB003 and ZAB006A, a further ELISA was performed using following peptides.

| | |
|---|---|
| human N-pro | SEQ ID NO:5 |
| N-pro30 | SEQ ID NO:6 |
| N-proIAPP | SEQ ID NO:3 |
| IAPP | SEQ ID NO:4 |

R1099 antibody (ascities, Abcam) was used as a control.

The results are shown in FIG. 5. ZAB003 was found to recognise N-pro, N-pro30 and N-proIAPP but not IAPP. ZAB006 was found to recognise N-pro30 and to a much lesser degree N-pro, which indicates that this epitope overlaps the N-terminal cleavage site of IAPP. ZAB006 showed little or no binding to NproIAPP. R1099 binds both NproIAPP and mature IAPP. These results show that ZAB003 can be used to specifically target the unprocessed or misprocessed form of IAPP without affecting the mature IAPP peptide.

### Example 3

### Biacore Experiments to Determine Monoclonal Antibody Affinity

Surface plasmon resonance was used to determine the binding affinity of monoclonal antibodies ZAB003 and ZAB006 to N-pro30.

Ligands (5 mg/ml ZAB003, 5 mg/ml ZAB006 or 5 mg/ml Npro30) were covalently bound to the CM5 sensor chip surface (Biacore, BR-1003-99) via amine groups using the Amine Coupling Kit (Biacore, BR-1000-50). ZAB003 and ZAB006 were diluted to 50 ug/ml and Npro30 to 200 ug/ml in 10 mM sodium acetate buffer (Biacore, BR-1003-51). The chip surface was activated for 8 minutes with a 1:1 mixture (v/v) of EDC and NHS at 5 ul/ml. Ligands were immobilised over 8 minutes at 5 ul/min. Excess reactive groups were deactivated with 1M ethanolamine, pH 8.5 for 8 minutes at 10 ul/min. ZAB003 and ZAB006 were serially diluted 1:1 from 5 uM to 19.5 nM. Each dilution was injected for 4 minutes over the Npro30 coated lane, followed by a 40 minute wash with PBS (10 mM phosphate, 2.7 mM KCl and 137 mM NaCl, pH 7.4). The sensor chip surface was regenerated with a 4 minute wash with 10 mM Glycine pH 1.5 (Biacore, BR-1003-54). The highest concentrations of antibody were also run over the blank lane to determine non-specific binding.

Data from the titration curves was analysed using BiaEvaluation software and fitted to a bivalent model appropriate for antibody binding kinetics.

No non-specific binding was found for either ZAB003 or ZAB006. ZAB006 exhibited an equilibrium binding affinity constant, K_{A}, of 2.2×10⁷ and a K_{D} of 4.5×10⁻⁸, while ZAB003 had a K_{A}, of 2.6×10¹⁰ and a K_{D} of 3.8×10⁻¹¹. These results show that ZAB003 binds with a higher affinity than ZAB006.

### Example 4

### Binding of Monoclonal Antibodies in the Presence of Heparin

To determine the binding of monoclonal antibody (moAb) to N-Pro30 to in the presence of heparin, the N-Pro30 can be preincubated with a saturating concentration of heparin sodium salt prior to binding of moAb. A goat anti-mouse HRP or any other suitable conjugate can then be used for detection. The N-Pro30 peptide may have an N-terminal biotin tag, so that a streptavidin-HRP conjugate can be used.

The effect of the selected monoclonal antibodies on the binding of N-Pro30 to heparin can be assessed using SEC and a heparin column, based on previously published methods, but with the addition of the moAb to the protocol (Park and Verchere, 2001). Briefly, N-Pro30 or N-Pro30 plus a saturating concentration of monoclonal antibody is pumped through a heparin column and the retention of N-Pro30 measured by absorbance at 216 nm and by western blot analysis of collected fractions.

### Example 5

### Seeding of hIAPP Aggregation by N-Pro Extended h-IAPP

Aggregation studies examining the seeding of hIAPP aggregation by N-Pro extended h-IAPP (SEQ ID NO: 3) (optionally bound to heparin) can be monitored using Thioflavin-T (Thio-T) binding experiments. The effect of the moAb on the interaction of N-Pro extended h-IAPP (SEQ ID NO: 3) with heparin and hence the seeding of hIAPP can also be measured using this method. As an example, heparin can be coated on a microwell plate, washed, then plates are incubated in the presence of N-Pro extended h-IAPP (SEQ ID NO: 3) to allow binding to the heparin. Finally, mature hIAPP is added to the plate and its aggregation time course measured using emission of fluorescence from the Thioflavin-T probe, which is sensitive to amyloid aggregation. N-Pro extended h-IAPP (SEQ ID NO: 3) preincubated with monoclonal antibody can also be exposed to the heparin coated surface of the plate and then the aggregation profile of added mature hIAPP can be followed. Antibodies that inhibit the interaction between of N-Pro extended h-IAPP (SEQ ID NO: 3) and heparin will be expected to block the nucleation of amyloid formation of mature hIAPP on the heparin surface.

The effect of NproIAPP on the aggregation of hIAPP in the absence of heparin was measured using Thioflavin-T fluorescence. NproIAPP (custom synthesis, Bachem) was dissolved in 100% HFIP (Sigma) aliquoted and speedvac'd until dry and then resuspended in sodium bicarbonate coating buffer 50 mM, pH 9.6 to 10 μg/ml. 384 well black optical bottom microwell plates (Nunc) were coated with 100 μl of either NproIAPP or coating buffer alone overnight at 4° C. Plates were then washed with 3×100 μl 50 μM Tris pH 7.5. Thioflavin T (Sigma) was made as a stock solution of 2.5 mM in ddH₂O filtered through a 0.2 μm filter (Millipore) and stored at −80° C. Immediately before use, the stock Thio-T was thawed and diluted in 50 mM Tris buffer pH 7.5 to achieve a final concentration of 65 μM. This solution was filtered through a 0.2 μm filter. All surrounding wells were filled with 90 μl of Thio-T buffer. hIAPP was resuspended to either 0.5 or 1 mg/ml in Thio-T buffer. Serial dilutions were made in Thio-T buffer and 90 ul was used in each well. An acetate sheet was used to cover the wells containing samples and a plate sealer applied on top. The assay was run using a Synergy HT plate reader (Biotek) set at 37° C. and a sensitivity of 65. Readings were taken every 10 minutes for 48 hours with a 2 second shake immediately before each time point. Data was exported into Excel and plotted. Each curve was normalized to 1 and the average taken of the replicates.

FIGS. 6 and 7 show that the aggregation profile of hIAPP varies with concentration. Lower concentrations of hIAPP result in longer lag phases and slower kinetics. In the presence of NproIAPP the lag time is shortened and the rate of aggregation is increased to a similar extent over a range of concentrations. Aggregation assays done with NproIAPP only (no hIAPP) did not show any increase in Thio-T signal and therefore any increase in signal is due to the hIAPP. These data show that NproIAPP is capable of seeding the aggregation and amyloid formation of hIAPP.

### Example 6

### Determination of Monoclonal Antibody Titre in Plasma of Mice Following Administration to Evaluate their Clearance. (Pharmacokinetics)

ZAB003 and ZAB006 antibodies were tested on groups of 3 mice (total 15 mice C57BL/6J background strain). A single subcutaneous (s.c.) injection of mAb (˜2 mg/kg dose) is given to each animal. Blood samples are taken daily for at least 1 week, T=0, day 1, day 2, etc. Sample plasma was analysed for circulating antibody levels and the rate of clearance determined.

Mouse plasma was supplied from day 0 to day 7 and each sample screened by ELISA against Npro30 peptide antigen. A 96 well Immulon maxisorp ELISA plate was coated with Npro30 peptide at 100 ng/well, overnight at 4° C. in Sodium Bicarbonate pH 9.2. The ELISA plate was washed 3 times with wash buffer (PBS/0.05% Tween 20 pH 7). A standard curve was prepared from 2 ug/ml double diluted ten times to 0.002 ug/ml, of the antibody used to inject the mouse whose plasma was being tested. Plasma samples were diluted 1:50, 1:150 and 1:300 into blocking buffer and added to the ELISA plate at 100 ul/well. The plate was then incubated for 1 hour at room temperature (RT) and washed 5 times with wash buffer. Goat anti-mouse HRP conjugated (Sigma) was diluted in blocking buffer (3% BSA/PBS/0.05% Tween 20 pH 7.4) and added to the plate at 100 ul/well. The plate was incubated for 1 hour at RT and washed 5 times with wash buffer. The ELISA was developed by adding TMB substrate (Sigma) at 100 ul/well and stopped after 5 minutes by addition of 25 ul Stop buffer (2M Sulphuric acid). Absorbance was read at 450 nm.

In all cases, the 1 in 50 dilution of plasma gave values above the standard curve. Half life values were therefore determined by averaging the data from the 1 in 150 and 1 in 300 dilutions for three mice per antibody group. FIG. 8 shows the changes in antibody level in one mouse for each antibody group and shows how the circulating antibody levels peak between 1 and 2 days post injection. The levels then drop off over the next few days.

The clearance rate combined with affinity to Npro30 peptide and binding in the presence of heparin data may be used to select the optimal antibody for use in animal trials.

### Example 7

### Treatment of Male hIAPP Transgenic Mice with Monoclonal Antibodies. Antibody Testing on ob/ob hIAPP(+/+) Mice (Hoppener, 1999)

4 groups of 16 mice were studied; a total of 48 transgenic and 16 non-transgenic mice were required:

Group 1. hIAPP transgenic s.c. injected with vehicle only
Group 2. hIAPP transgenic s.c. injected with ZAB003
Group 3. hIAPP transgenic s.c. injected with ZAB006
Group 4. 15 non-transgenic ob/ob mice (ob/ob: homozygous for the ‘obese’ mutation) injected with vehicle only

Monoclonal antibodies were injected at 5 mg/kg once per week as determined by the clearance rate described above. Antibody administration was commenced at 8-10 weeks of age (or as soon as s.c. injections can be started without risk to the animal).

Plasma samples from the eight week bleed were screened by ELISA for circulating antibody levels using Npro30 peptide antigen as described above.

FIGS. 9 and 10 show that there is circulating antibody present in the plasma samples from these two small treatment groups eight weeks after the start of the trial protocol. The mice are actually bled seven days post injection and the higher levels of ZAB006 (compared to ZAB003) may be due to its slightly longer half life; as determined in the clearance study.

Antibody administration continues for up to 10 months or until the observed differences between treated and untreated animals suggest that the experiment be interrupted. Levels of circulating antibody are measured regularly to detect anti-idiotypic responses and ascertain the real efficacy of the treatment only on those animals in which the monoclonal antibody is not cleared and therefore remains available for binding to the endogenous N-terminus extended forms of hIAPP.

Progression towards diabetes in the mice is monitored in accordance with the protocol set forth in Example 9 below.

### Example 8

### Treatment of hIAPP Transgenic Mice with Monoclonal Antibodies. Antibody Testing on Avy/a hIAPP(+/+) or (+/−) Mice (Soeller, 1998)

4 groups of 16 mice are studied; a total of 48 transgenic and 16 non-transgenic mice are required:

Group 1. hIAPP transgenic s.c. injected with vehicle only
Group 2. hIAPP transgenic s.c. injected with ZAB003
Group 3. hIAPP transgenic s.c. injected with ZAB006
Group 4. 16 non-transgenic Avy/a mice (Avy/a: heterozygous insulin-resistant agouti viable yellow strain) injected with vehicle only.

Selected monoclonal antibodies are injected at 5 mg/kg at weekly intervals, as determined by the clearance rate (see example 3 above).

Antibody administration is commenced at 6-8 weeks of age (or as soon as s.c. injections can be started without risk to the animal) and continue for up to 10 months, or until the observed differences between treated and untreated animals suggest that the experiment be interrupted.

The first injection is administered intravenous (i.v.) and is followed by sub cutaneous weekly doses thereafter. The first i.v. administration is included to increase the tolerance of the mice to the antibody and reduce the risk of anti-idiotypic responses. Levels of circulating antibody are measured regularly to detect anti-idiotypic responses and ascertain the real efficacy of the treatment only on those animals in which the monoclonal antibody is not been cleared and therefore it remains available for binding to the endogenous N-terminus extended forms of hIAPP.

Progression towards diabetes in the mice is monitored in accordance with the protocol set forth in Example 9 below.

### Example 9

### Examination of Diabetic Status of Mice and Amyloid Load

Every two weeks and before dosing with the monoclonal antibodies each animal is bled. The following tests are performed on each blood sample:

1. Determination of glucose levels in plasma.
2. Determination of insulin levels in plasma.
3. Determination of IAPP levels in plasma (as appropriate, for example every four to eight weeks)
4. Determination of monoclonal antibody titre in plasma.

The fasting plasma glucose level was determined in the following groups of treated mice at T=8 weeks of ZAB003 and ZAB006 antibody administration.

Group 1 ob/ob, hIAPP transgenic, vehicle only (4 mice)
Group 2 ob/ob, hIAPP transgenic, ZAB003 (5 mice)
Group 3 ob/ob, hIAPP transgenic, ZAB006 (5 mice)
Group 4 ob/ob, non-transgenic, vehicle only (4 mice)

A fasting plasma sample (fasted 12-15 hours) was obtained via orbital puncture. Glucose levels were determined in the plasma using the VITROS 250 machine (ORTHO Diagnostic). FIG. 11 shows the average glucose levels determined for each group. The data indicates that the group treated with ZAB003 has lower glucose levels than the untreated group while mice treated with ZAB006 may have slightly higher fasting plasma glucose.

The measurements detailed above (points 1-4) are used to monitor progression towards diabetes in the mice (samples will be taken prior to antibody dosing as detailed in examples 7 and 8). The endpoint of the experiment is the point at which untreated transgenic animals have increased plasma IAPP and insulin levels compared to T=0 measurements and are hyperglycemic. The non-transgenic ob/ob mice at this point should also have increased levels of IAPP and insulin but a normal glucose level. (Soeller, 1998; Hoppener, 1999).

One week after the last dose of MoAb, a Glucose tolerance test (IPGTT) is performed. Subsequently, the animals are sacrificed and a terminal bleed is collected. All pancreata are excised and stored appropriately for immunohistochemical analysis. Other organs may also be stored for comparative examination.

The final bleed is analysed for:

1. Levels of glucose, Insulin, IAPP, and monoclonal antibody titre
2. Level of glycated haemoglobin (HbA1a)
3. Detection of markers of amyloid disease, such as amount of amyloid plaques or early aggregated species.
4. Pancreata are analysed by optical and electron microscopy to monitor amyloid load and islet degeneration.

### Sequence Listing Information

SEQ ID NO: 1. N-Pro-KC (N-Pro fragment of proIAPP plus first two residues of mature hIAPP)

TPIESHQVEKRKC

SEQ ID NO: 2. ProIAPP (Including N-Pro and C-Pro hIAPP flanking regions). A disulfide bridge is present between the two cysteine residues

TPIESHQVEKRKCNTATCATQRLANFLVHSSNNFGAILSSTNVGSNTYGK
RNAVEVLKREPLNYLPL

SEQ ID NO: 3. N-Pro-hIAPP. A disulfide bridge is present between the two cysteine residues

TPIESHQVEKRKCNTATCATQRLANFLVHSSNNFGAILSSTNVGSNTY-
NH2

SEQ ID NO: 4. Mature hIAPP. A disulfide bridge is present between the two cysteine residues

KCNTATCATQRLANFLVHSSNNFGAILSSTNVGSNTY-NH2

SEQ ID NO: 5. N-Pro peptide.

TPIESHQVEKR

SEQ ID NO: 6. N-Pro30. N-terminus extended hIAPP fragment as described by Park and Verchere (2001). A disulfide bridge is present between the two cysteine residues

TPIESHQVEKRKCNTATCATQRLANFLVHS

SEQ ID NO: 7 Mouse Npro peptide

TPVRSGSNPQMDKR

### SEQ ID NO: 8 Mouse Npro16

TPVRSGSNPQMDKRKC

### REFERENCES

Badman, M. K et al. FEBS Lett 378, 227-31, 1996.
Butler, A. E., et al Diabetes 52, 102-10, 2003a.
Butler, A. E., et al Diabetes 52, 2304-14, 2003b.
Higham, C. E. et al European Journal of Biochemistry 267:4998-5004, 2000.
Hoppener, J. W. M. et al Diabetologia 42, 427-434, 1999.
Hou, X., et al Diabetologia 42:188-194, 1999.
Janson, J. et al Proc Natl Acad Sci USA 93, 7283-8, 1996.
Janson, J. et al Diabetes 48, 491-498, 1999.
Kayed, R. et al Science 300, 486-9, 2003.
Lorenzo, A. et al Nature 368, 756-60, 1994.
MacGibbon, G. A. et al Neuroreport 8, 3945-50, 1997.
Marzban, L. et al Diabetes 53:141-148, 2004.
Park, K., and Verchere, C. B. J. Biol. Chem 276 (20), 16611-16616, 2001.
Saafi, E. L. et al Cell Biol Int 25, 339-50, 2001.
Soeller, W. C. et al Diabetes 47, 743-750, 1998.
Tucker, H. M. et al J Neurochem 71, 506-16, 1998.
Wang, J. et al Diabetes 50:534-539, 2001.
Watson, D. J. et al J Biol Chem 272, 31617-24, 1997.
Westermark, P. et al Diabetes Research and Clinical Practice 7:219-226, 1989.
Zhang, S. et al FEBS Lett 455, 315-20, 1999.

1-48. (canceled)

49. A method for the prevention or treatment of type 2 diabetes comprising administering an agent which is capable of binding specifically to one or more of soluble proIAPP (SEQ ID NO: 2), N-Pro-hIAPP (SEQ ID NO: 3), or peptide fragments containing the N-Pro sequence (SEQ ID NO: 5) to an individual in need thereof.

50. The method of claim 49, wherein the agent comprises a peptide, protein, domain, antibody or antibody fragment capable of binding specifically to one or more of soluble proIAPP (SEQ ID NO: 2), N-Pro-hIAPP (SEQ ID NO: 3), or peptide fragments containing the N-Pro sequence (SEQ ID NO: 5).

51. The method of claim 49, wherein the agent which binds specifically to N-Pro-hIAPP (SEQ ID NO: 3).

52. The method of claim 49, wherein the agent is capable of binding specifically to an epitope on the heparan sulphate binding site of a polypeptide comprising the N-Pro sequence (SEQ ID NO: 5).

53. The method of claim 49, wherein the agent is an antibody or fragment thereof.

54. The method of claim 53, wherein the agent wherein the antibody is a monoclonal antibody.

55. The method of claim 53, wherein the antibody is selected from the group consisting of: a human antibody, a rodent antibody, a murine antibody, a camelid antibody, a recombinant human antibody, a humanised murine antibody, a chimerised murine antibody, a transgenic murine antibody and a chimerised or humanised camelid antibody.

56. The method of claim 49, wherein the agent is capable of binding specifically to a soluble protein or peptide consisting of an amino acid sequence selected from:
a) the amino acid sequence of SEQ ID NO: 1;
b) residues 1-11 of the sequence of SEQ ID NO: 1 (SEQ ID NO: 5);
c) fragments of 5-8 amino acids of the N-terminal sequence of SEQ ID NO: 1;
d) fragments of 5-8 amino acids of the sequence of SEQ ID NO: 1 including His6, Lys10 Arg11 and Lys12 and
(e) fragments of 5-8 amino acids of the sequence of SEQ ID NO: 3 which include one or more residues of SEQ ID NO:5.

57. The method of claim 49, wherein the agent is capable of stimulating production of an immune response in an individual to whom the agent has been administered, said immune response being capable of inhibiting specifically the interaction between soluble polypeptides comprising the N-Pro sequence (SEQ ID NO: 5) and a basement membrane heparan sulphate proteoglycan.

58. The method of claim 57, wherein the agent comprises a soluble peptide or protein consisting of an amino acid sequence selected from:
a) the amino acid sequence of SEQ ID NO: 1;
b) residues 1-11 of the sequence of SEQ ID NO: 1 (SEQ ID NO: 5);
c) fragments of 5-8 amino acids of the N-terminal sequence of SEQ ID NO: 1;
d) fragments of 5-8 amino acids of the sequence of SEQ ID NO: 1 including His6, Lys10 Arg11 and Lys12 and
(e) fragments of 5-8 amino acids of the sequence of SEQ ID NO: 3 which include one or more residues of SEQ ID NO:5.

59. The method of claim 49, wherein the agent comprises an amino acid sequence selected from:
a) the amino acid sequence of SEQ ID NO: 1;
b) residues 1-11 of the sequence of SEQ ID NO: 1 (SEQ ID NO: 5);
c) fragments of 5-8 amino acids of the N-terminal sequence of SEQ ID NO: 1;
d) fragments of 5-8 amino acids of the sequence of SEQ ID NO: 1 including His6, Lys10 Arg11 and Lys12; and
(e) fragments of 5-8 amino acids of the sequence of SEQ ID NO: 3 which include one or more residues of SEQ ID NO:5
fused to another peptide sequence.

60. The method of claim 59, wherein the other peptide sequence is an anti-amyloidogenic agent.

61. The method of claim 49, wherein the agent comprises a structural mimic of an epitope of a peptide consisting of an amino acid sequence selected from:
a) the amino acid sequence of SEQ ID NO: 1;
b) residues 1-11 of the sequence of SEQ ID NO: 1 (SEQ ID NO: 5);
c) fragments of 5-8 amino acids of the N-terminal sequence of SEQ ID NO: 1;
d) fragments of 5-8 amino acids of the sequence of SEQ ID NO: 1 including His6, Lys10 Arg11 and Lys12 and
(e) fragments of 5-8 amino acids of the sequence of SEQ ID NO: 3 which include one or more residues of SEQ ID NO:5.

62. The method of claim 61, wherein the agent is a peptide mimic of an epitope present on a peptide having an amino acid sequence selected from:
a) the amino acid sequence of SEQ ID NO: 1;
b) residues 1-11 of the sequence of SEQ ID NO: 1 (SEQ ID NO: 5);
c) fragments of 5-8 amino acids of the N-terminal sequence of SEQ ID NO: 1; and
d) fragments of 5-8 amino acids of the sequence of SEQ ID NO: 1 including His6, Lys10 Arg11 and Lys12 and
(e) fragments of 5-8 amino acids of the sequence of SEQ ID NO: 3 which include one or more residues of SEQ ID NO:5.

63. The method of claim 49, wherein the agent is an anti-idiotypic antibody.

64. A method for the prevention or treatment of type 2 diabetes comprising administering an agent capable of inhibiting the interaction between soluble molecules corresponding to unprocessed or partially processed forms of proIAPP (SEQ ID NO: 2) that include the sequence N-Pro (SEQ ID NO: 5) or fragments thereof, and a heparan sulphate proteoglycan to an individual in need thereof.

65. The method of claim 64, wherein the agent is capable of binding specifically to molecules that include the sequence N-Pro (SEQ ID NO: 5) or fragments thereof.

66. The method of claim 65 wherein the agent comprises, a protein, peptide, antibody or antibody fragment capable of binding specifically molecules that include the sequence N-Pro (SEQ ID NO: 5) or fragments thereof.

67. The method of claim 64 wherein the agent is capable of binding specifically to molecules that include the sequence N-Pro (SEQ ID NO: 5) or fragments thereof, and thereby inhibiting specifically the interaction, of soluble molecules corresponding to unprocessed or partially processed forms of proIAPP (SEQ ID NO: 2) that include the sequence N-Pro (SEQ ID NO: 5) or fragments thereof with heparin or heparan sulphate.

68. The method of claim 64, wherein the agent comprises an antibody or fragment thereof capable of binding specifically to an epitope included in the N-terminal region of soluble N-Pro (SEQ ID NO: 3).

69. The method of claim 68, wherein the agent comprises a monoclonal antibody or fragment thereof.

70. The method of claim 64, wherein the agent consists of or comprises an antibody or a fragment thereof, preferably a monoclonal antibody or fragment thereof, capable of binding specifically a peptide derived from the N-terminal region of soluble N-Pro (SEQ ID NO: 3).

71. The method of claim 70, wherein the agent consists of, or comprises, a monoclonal antibody or fragment thereof.

72. A method according to claim 64, wherein the agent is a monoclonal antibody or a fragment thereof capable of binding specifically an amino acid sequence selected from:
a) the amino acid sequence of SEQ ID NO: 1;
b) residues 1-11 of the sequence of SEQ ID NO: 1 (SEQ ID NO: 5);
c) fragments of 5-8 amino acids of the N-terminal sequence of SEQ ID NO: 1;
d) fragments of 5-8 amino acids of the sequence of SEQ ID NO: 1 including His6, Lys10 Arg11 and Lys12 and
(e) fragments of 5-8 amino acids of the sequence of SEQ ID NO: 3 which include one or more residues of SEQ ID NO:5.

73. A method for the prevention or treatment of type 2 diabetes, characterised by administration of an agent capable of stimulating production of an immune response capable of inhibiting specifically the interaction between soluble molecules corresponding to unprocessed or partially processed forms of proIAPP (SEQ ID NO: 2) that include the sequence N-Pro (SEQ ID NO: 5) or fragments thereof and a heparan sulphate proteoglycan.

74. The method of claim 73, wherein the agent is a peptide derived from the N-terminal domain of proIAPP (SEQ ID NO: 2) that includes at least in part N-Pro IAPP (SEQ ID NO: 5).

75. The method of claim 73, wherein the agent is a soluble peptide comprising, an amino acid sequence selected from:
a) the amino acid sequence of SEQ ID NO: 1;
b) residues 1-11 of the sequence of SEQ ID NO: 1 (SEQ ID NO: 5);
c) fragments of 5-8 amino acids of the N-terminal sequence of SEQ ID NO: 1;
d) fragments of 5-8 amino acids of the sequence of SEQ ID NO: 1 including His6, Lys10 Arg11 and Lys12 and
(e) fragments of 5-8 amino acids of the sequence of SEQ ID NO: 3 which include one or more residues of SEQ ID NO:5.

76. A pharmaceutical composition comprising an agent which is capable of binding specifically to one or more of soluble proIAPP (SEQ ID NO: 2), N-Pro-hIAPP (SEQ ID NO: 3), or peptide fragments containing the N-Pro sequence (SEQ ID NO: 5) and a pharmaceutically acceptable carrier or diluent.

77. The pharmaceutical composition of claim 76, further comprising an adjuvant.

78. The pharmaceutical composition of claim 76, formulated for administration by a route selected from the group consisting of intranasal, intradermal, subcutaneous, intramuscular, or intravenous administration.


SEQUENCE LISTING (9 sequences)

SEQ ID NO: 1 - protein, 13 aa, Homo sapiens
TPIESHQVEKRKC

SEQ ID NO: 2 - protein, 67 aa, Homo sapiens
  DISULFID (13)..(18)
TPIESHQVEKRKCNTATCATQRLANFLVHSSNNFGAILSSTNVGSNTYGKRNAVEVLKREPLNYLPL

SEQ ID NO: 3 - protein, 48 aa, Homo sapiens
  DISULFID (13)..(18)
  MOD_RES (48)..(48): AMIDATION
TPIESHQVEKRKCNTATCATQRLANFLVHSSNNFGAILSSTNVGSNTY

SEQ ID NO: 4 - protein, 37 aa, Homo sapiens [sequence omitted]

SEQ ID NO: 5 - protein, 11 aa, Homo sapiens
TPIESHQVEKR

SEQ ID NO: 6 - protein, 30 aa, Homo sapiens
  DISULFID (13)..(18)
TPIESHQVEKRKCNTATCATQRLANFLVHS

SEQ ID NO: 7 - protein, 14 aa, Mus musculus
TPVRSGSNPQMDKR

SEQ ID NO: 8 - protein, 16 aa, Mus musculus
TPVRSGSNPQMDKRKC

SEQ ID NO: 9 - protein, 89 aa, Homo sapiens
MGILKLQVFLIVLSVALNHLKATPIESHQVEKRKCNTATCATQRLANFLVHSSNNFGAILSSTNVGSNTYGKRNAVEVLKREPLNYLPL